(19) United States
(12) Patent Application Publication
(10) Pub. No.: US 2023/0001206 A1
(43) Pub. Date: Jan. 5, 2023

(54) HEARING PROSTHESIS SYSTEM

(71) Applicant: Advanced Bionics AG, Staefa (CH)

(72) Inventors: Joachim Thiemann, Hannover (DE); Raphael Koning, Wedemark (DE); Kanthaiah Koka, Valencia, CA (US)

(21) Appl. No.: 17/853,281

(22) Filed: Jun. 29, 2022

(30) Foreign Application Priority Data
Jul. 5, 2021 (EP) EP21183758

(51) Int. Cl. A61N 1/36; A61N 1/05; A61B 5/00

(52) U.S. Cl. CPC A61N 1/36038; A61N 1/0541; A61B 5/4851; A61N 1/36031

(57) ABSTRACT

A hearing prosthesis system may include a cochlear implant coupled to an electrode array and configured to be implanted within a patient; and a processing unit communicatively coupled to the cochlear implant which is configured to direct the cochlear implant to apply stimulation to a cochlea of the patient via the electrode array and to detect, via the electrode array, a neural response of the patient to hearing stimulation. The processing unit is further configured to generate a user interaction audio signal indicative of an interaction of the patient with the hearing prosthesis system and apply perceivable hearing stimulation to the patient according to the user interaction audio signal, and to record, via the electrode array and the cochlear implant, the neural response to said hearing stimulation according to the user interaction audio signal, thereby utilizing the user interaction audio signal as a test audio signal.

### RELATED APPLICATIONS

The present application claims priority to EP Patent Application No. EP21183758, filed Jul. 5, 2021, the contents of which are hereby incorporated by reference in their entirety.

### BACKGROUND INFORMATION

Hearing prosthesis systems may use electrical stimulation of the cochlea via the electrode array only or, for patients with residual hearing, the system in addition may apply acoustic stimulation (such bimodal stimulation systems are known as EAS-systems). Hearing prosthesis systems have to be adapted to the individual patient by fitting sessions, wherein fitting parameters, such as electrical stimulation or acoustic stimulation threshold levels, acoustic amplification gains for EAS-patients, etc., are determined and set. Since the condition of the patient's hearing and electrode conditions may vary over time, regular assessment of the performance of the hearing prosthesis system by monitoring the operating parameters is desirable. In particular, shifts of the steady state parameters should be determined and the normal viability thereof should be tracked.

Assessment of the system performance may include objective measurements, such as measurement of electrocochleography (ECochG) thresholds, neural response imaging (NRI) thresholds and cortical responses, via the electrode array, wherein the measured electrical signals are transmitted via back-telemetry to the external processing unit. Such measurements involve electrical stimulation via the electrode array and/or acoustic stimulation (in case of EAS systems). Such hearing stimulation often involves an audible or at least noticeable percept that is not consistent with normal stimulation pattern and therefore is considered unnatural by user. As a consequence, such system performance measurements typically are conducted only at visits of the patient to the hearing care professional (HCP), which may be infrequent and/or irregular.

WO 2020/044307 A1 relates to a hearing prosthesis system wherein stimulation for objective measurements of system performance is applied during times when a state of sleep of the patient has been determined, so as to avoid that the patient is disturbed by the hearing stimulation required for the objective measurements.

US 2017/0304632 A1 relates to a cochlear implant system wherein automated ECochG testing outside of a clinical setting is performed using the normal sounds that a patient hears in the course of the daily life. To this end, ambient sound signals received by the cochlear implant system during normal operation are analyzed to identify portions of sound signals that are suitable for performing an ECochG measurement using the implanted electrode array. The ECochG measurements are used for assessing the patient's residual hearing, so as to initiate corrective actions if a change of the residual hearing is detected.

### BRIEF DESCRIPTION OF THE DRAWINGS

Hereinafter, examples of the invention will be illustrated by reference to the attached drawings, wherein:

FIG. 1 is a schematic illustration of a hearing prosthesis system during a neural response measurement;

FIG. 2 is a schematic illustration of an example of a hearing prosthesis system;

FIG. 3 is a schematic illustration of an electrode array of the system of FIGS. 1 and 2, as inserted in a cochlea, during a neural response measurement; and

FIG. 4 is a schematic illustration of an example of user interactions resulting in a user interaction audio signal to be used in a neural response measurement.

### DETAILED DESCRIPTION

Described herein is a hearing prosthesis system comprising an implantable electrode array, a cochlear implant coupled to the electrode array, and a processing unit communicatively coupled to the cochlear implant.

In some embodiments, a hearing prosthesis system is provided which allows for relatively regular monitoring of the system performance in a patient-friendly manner.

The embodiments described herein may utilize a user interaction audio signal which is indicative of an interaction of the patient with the hearing prosthesis system as a test audio signal for applying perceivable hearing stimulation to the patient according to the user interaction signal and to detect, via the electrode array, a neural response of the patient to such hearing stimulation. Thereby objective system performance measurements can be conducted in an unobtrusive manner on a relatively frequent basis during normal use, in particular outside a clinical setting, of the hearing prosthesis system.

In some implementations, the user interaction audio signal is a feedback message signal indicative of a user interaction on a user interface of the hearing prosthesis system or a status message signal indicative of a change in a condition of the hearing prosthesis system. For example, the feedback message signal may be indicative of a user action resulting in a hearing program change, a volume increase, a volume reduction or locking or unlocking of the implant. For example, the status message signal may be indicative of a wireless connection or disconnection of the external processing unit with external device, or it may be indicative of a low battery status.

In some implementations, the user interaction audio signal is selected such that is perceivable by the patient as a standard sound associated with the respective feedback message or a status message.

In some implementations, the feedback message signal is in response to a user action on an operator control. For example, the operator control may be a mechanical element disposed on an external housing including the processing unit; for example, the operator control may be a button or a key to be pressed. According to another example, the operator control may be provided on a remote control device communicatively coupled with the processing unit; for example, the operator control may be implemented as a touch screen of the remote control device. According to still another example, the feedback message signal may be in response to a user action on an operator control of an accessory device communicatively coupled to the processing unit; for example, the accessory device may be smartphone and the user action may be locking or unlocking of a touchscreen of the smartphone.

In some implementations, the processing unit is configured to apply the perceivable hearing stimulation corresponding to the user interaction audio signal at a level within the comfort range.

In some implementations, the user interaction audio signal includes sinusoidal tones and/or frequency sweeps.

In some implementations, the recording of the neural response to the user interaction audio signal includes neural response imaging (NRI) threshold measurements, electrocochleography (ECochG) threshold measurements, cortical response measurements and/or electrode impedance measurements.

In some implementations, the processing unit is configured to apply said perceivable hearing stimulation to the patient according to the user interaction audio signal as electrical stimulation via the electrode array. For example, the processing unit may be configured to select only some of the electrodes of the electrode array which are found to characterize the fitting curves and to use only the selected electrodes for the electrical stimulation according to the user interaction audio signal.

In some implementations, the hearing prosthesis system is an EAS system including an electroacoustic output transducer, wherein the processing unit is configured to apply said perceivable hearing stimulation to the patient according to the user interaction audio signal as acoustic stimulation via the electroacoustic output transducer. Fore example, the recording of the neural response to the user interaction audio signal may include measurement of ECochG signals.

In some implementations, the processing unit is configured to automatically adjust fitting parameters of the hearing prosthesis system according to the recorded neural response. For example, the processing unit may be configured to automatically adjust stimulation threshold levels of the hearing prosthesis system according to the recorded neural response, and wherein the user interaction audio signal may result in hearing stimulation suitable for determining the respective threshold from the recorded neural response; for example, the recording of the neural response to the user interaction audio signal may include cortical response measurements for adjustment of electrical stimulation thresholds.

In some implementations, the processing unit is configured to store data corresponding to neural responses recorded over a time period for later use in manual adjustment of fitting parameters.

In some implementations, the processing unit is integrated an external unit. For example, the external unit may be configured to be worn at the patient's head; for example, the external unit is a BTE unit or a headpiece.

In some implementations, the cochlear implant and the processing unit are coupled via an inductive transcutaneous link, wherein the neural response to the user interaction audio signal is supplied to the processing unit by back telemetry via the inductive transcutaneous link.

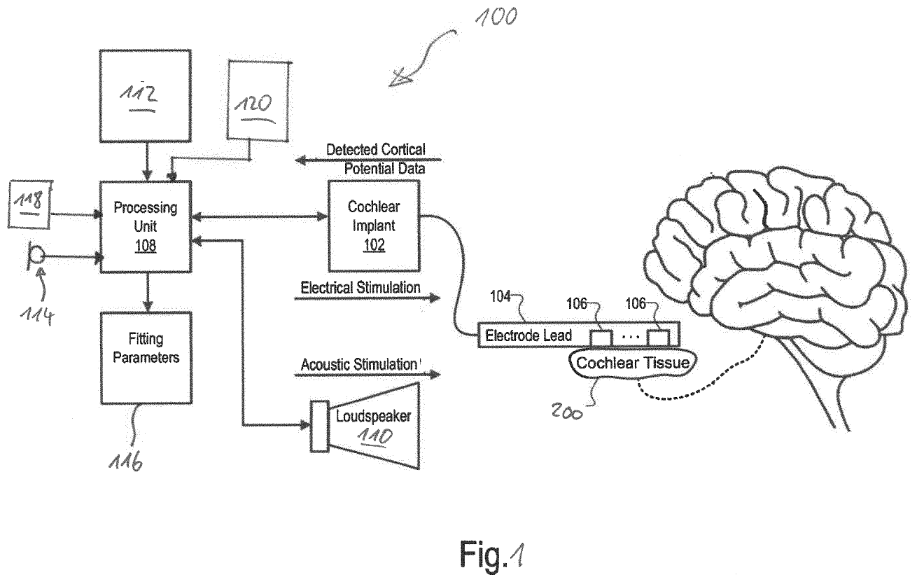

FIG. 1 is a schematic illustration of functional components of an example of a cochlear implant system 100, comprising a cochlear implant 102 to be implanted within a patient, an electrode array (or electrode lead) 104 comprising a plurality of electrodes 106 to be implanted within a cochlea 200 of the patient and coupled to the cochlea implant 102, and a processing unit 108 communicatively coupled to the cochlea implant 102. The system may further comprise an electroacoustic output transducer 110, such as a loudspeaker, coupled to the processing unit 108 for providing acoustic stimulation to the patient's hearing, a user interface 112 coupled to the processing unit 108, a microphone 114 coupled to the processing unit 108 for capturing input audio signals from ambient sound, and a memory 116 coupled to the processing unit 108 for storing fitting parameters and other system parameters.

The processing unit 108 is configured to direct the cochlear implant 102 to apply stimulation to the cochlea 200 via the electrodes 106 of the electrode array 104 and to direct the electroacoustic output transducer 110 to apply acoustic stimulation to the patient so as to stimulate the residual hearing of the patient. To this end, the processing unit 108 generates corresponding electrical stimulation signals which are supplied to the cochlear implant 102 and acoustic stimulation signals which are supplied to the electroacoustic output transducer 110. The processing unit 108 may generate such stimulation signals based on audio input signals received from the microphone 114 or an audio input 118 which may be, for example, a communication interface to an accessory device, such as a smartphone or an audio streaming device, so as to stimulate, in a normal operation mode, the hearing of the patient according to the input audio signals to make the input audio signals perceivable by the patient.

Further, the processing unit 108 may generate electrical and/or acoustic stimulation signals in response to a user interaction with the user interface 112, so as to provide a feedback message, such as a characteristic tone, to the patient, and/or the processing unit 108 may generate the stimulation signals according to a status change of the system, such as a wireless connection or disconnection of the processing unit with an external device, such as a smartphone, or to indicate a low battery status. To this end, the processing unit 108 may be coupled to respective sensors 120 which sense certain system conditions such as battery status.

Electrical stimulation via the electrodes 106 and/or acoustic stimulation or via the output transducer 110 results in a neural response of the patient to such hearing stimulation, which neural response can be detected via the electrode 106 of the electrode array, with the detected neural response signals being supplied via the cochlea implant 102 to the processing unit 108 for analysis of neural response signals.

As already mentioned above, the processing unit 108 may generate a user interaction audio signal which is indicative of an interaction of the patient with the hearing prosthesis system 100, and apply perceivable hearing stimulation to the patient according to the user interaction audio signal. The user interaction audio signal, for example, may be a feedback message signal indicative of a user action, in particular an action by the patient, on the user interface 112 or a status message signal indicative of a change in a condition of the hearing prosthesis system, such as sensed by the sensors 120.

The processing unit 108 further may record, via the electrode array 104 and the cochlear implant 102, the neural response to such hearing stimulation according to such user interaction audio signal, thereby utilizing the user interaction audio signal as a test audio signal. In this way hearing prosthesis performance can be monitored by performing relatively frequently active objective neural response measurements in a relatively unobtrusive way, i.e. without disturbing the patient by perception of unexpected or unusual hearing sensations, so that implant health and operation parameter fitting can be monitored during normal use of the hearing prosthesis by the patient. In particular, the test audio signals thereby can be designed in a manner that the patient experiences the expected perception of user interface feedback and/or status information. In other words, the patient perceives usually expected standard sounds/signals only. In particular, any signal tone may consist of suitable measurement stimuli for purposes such as fitting of the electrical and/or acoustical path. In other words, a user interaction with the hearing prosthesis actually triggers an active measurement with a stimulus at a level to be clearly perceivable, while the patient actually does not recognize that a neural response measurement presently takes place.

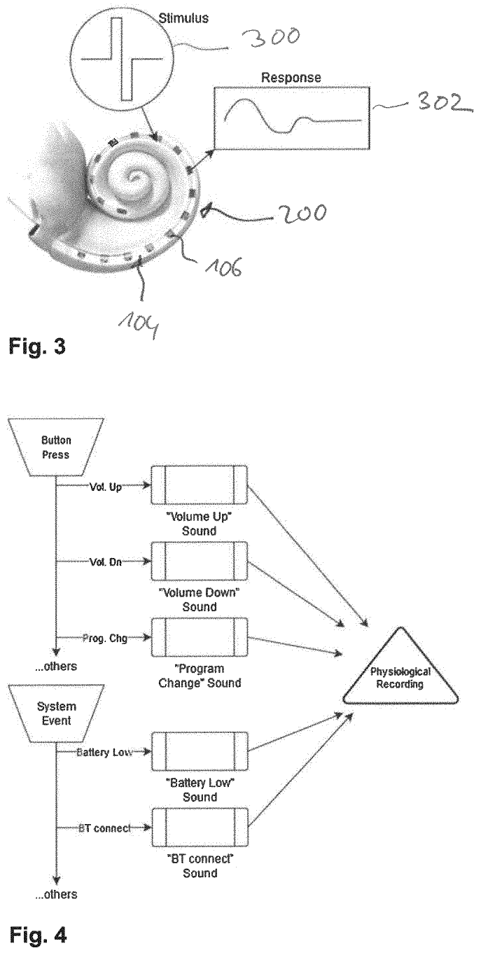

FIG. 3 is an illustration of a neural response measurement, wherein the electrode array 104 with electrodes 106 is shown as implanted within a cochlea 200. An example of a stimulation signal applied to an electrode 106 is schematically shown at 300 and a corresponding neural response signal collected by another one of the electrodes 106 in response to the stimulation signal 300 as indicated at 302.

The perceivable hearing stimulation corresponding to the user interaction audio signal may be applied at a level within the comfort range of the patient.

In some examples, the user interaction audio signals may include sinusoidal tones and/or frequency sweeps.

In some implementations, known previously used measurement signals may be modified so as to be clearly audible to the patient and create a pleasant perception which is similar or equal to a standard sound perception typical for user interaction with the hearing prosthesis system. Further, known previously used common user interaction audio signals, such as user interface feedback sounds, may be modified so as to be suitable for performing neural response measurements, while still being perceivable as a standard sound. Generally, also new stimuli may be designed, as long as the patient feels to perceive some kind of standard user interaction sound, while the patient should not have the feeling of a measurement being performed.

Examples of interactions of the patient with the hearing prosthesis system are schematically shown in FIG. 4, according to which user interaction with the user interface 112 may include “volume up”, “volume down” or “program change” commands by the patient, resulting in the processing unit 108 generating corresponding user feedback sounds. Another example is an implant lock or unlock command by the patient. For example, the “implant lock” sound may be a tone sequence resulting from stimulation on selected ones of the electrodes 106. A “volume up” sound may include ascending measurements on even-numbered electrodes, and a “volume down” sound may comprise descending measurements on odd-numbered electrodes. According to a further example, a “program change” sound may be a long tone resulting from stimulation on a selected electrode depending on the selected program. In all cases, the current level may depend on the presently selected volume, which, in case of “volume up” or “volume down” commands may be the present volume level or the new volume level. Examples of system events resulting in a status message signal may be “battery low”, resulting in a characteristic “battery low” sound, or “Bluetooth connect” resulting in a corresponding characteristic “Bluetooth connect” sound.

According to another example, the interaction of the patient with the user interface 112 may be the locking or unlocking of a screen of a smartphone communicatively coupled, for example, via a Bluetooth connection, to the processing unit. In this case, the processing unit 108 may perform, for example, an electrode impedance measurement when such unlocking or locking of the smartphone screen happens, which will cause a noticeable short sound which equals or is similar to a sound typically used for indicating screen locking/unlocking. The processing unit 108 then may verify that the resulting impedance measurements are within the expected range or, alternatively or in addition, it may store the measurement values in the memory 116 for long-term trend analysis.

In some implementations, the feedback message signal may be in response to a user interaction on an operator control, which, for example, may be a mechanical element disposed on an external housing including the processing unit 108; in particular, the operator control may be a button or a key to be pressed. In another example, the operator control may be provided on a remote control device communicatively coupled with the processing unit 108; for example, the operator control in this case may be implemented as a touchscreen of the remote control device. In some implementations, the feedback message signal may be in response to a user action on an operator control of an accessory device coupled to the processing unit 108; in particular, the accessory device may be a smartphone and the user action may be locking or unlocking of a touchscreen of the smartphone, as already mentioned above.

The recording of the neural response to the user interaction audio signal may include NRI threshold measurements, ECochG threshold measurement, cortical response measurements and/or electrode impedance measurements.

For applying electrical stimulation according to the user interaction audio signal the processing unit 108 may select some of the electrodes 106 of the electrode array 104, namely those electrodes which are found to characterize the fitting curves of the hearing prosthesis system. Accordingly, it is sufficient to use only these selected electrodes for the electrical stimulation according to the user interaction audio signal, i.e., for the neural response measurements; there is no need to use all electrodes.

In case that the hearing prosthesis system provides also for acoustic stimulation, measurement of ECochG signals is particularly useful.

For example, an acoustic sound could be presented via the output transducer 110 at different frequencies and the resulting ECochG signals may be measured via the electrodes 106. With the measured values the acoustic threshold could be estimated and compared to the present setting, and subsequently could be adjusted automatically if required.

Electrical stimulation via the electrodes 106 may be used for evoking and measure cortical potentials. Such cortical potential measurements can be used for validating the electrical threshold levels and to automatically adjust the levels if required, as described, for example, in U.S. patent application 62/926,351, which application is incorporated herein by reference in its entirety.

In general, the recorded neural responses may be used by the processing unit 108 to automatically adjust all kinds of fitting parameters, not only threshold levels.

Alternatively or in addition, the processing unit 108 may store data corresponding to the neural responses recorded over a time period in the memory 116 for later use in manual adjustment of fitting parameters, for example, by a hearing care professional.

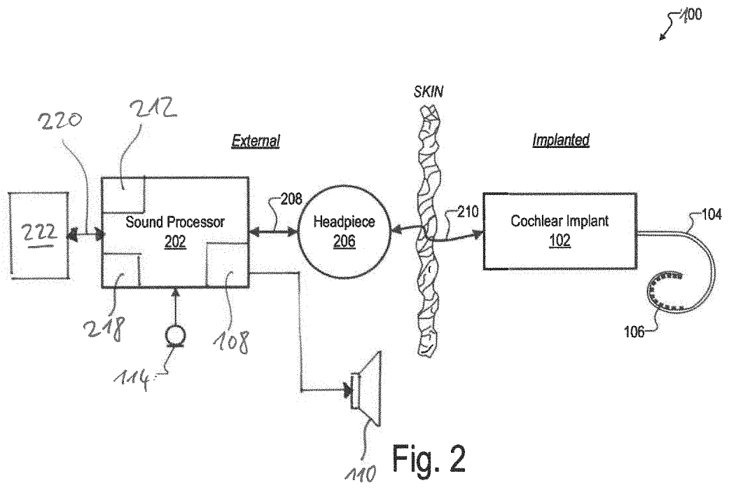

An example of a practical implementation of the system illustrated in FIG. 1 is schematically shown in FIG. 2, wherein the processing unit 108 forms part of an external unit, which in the example of FIG. 2 is formed by a sound processor 202 which also may include a wireless interface 218 for establishing a wireless connection 220 to an external device 222, such as a smartphone. The sound processor 202 further may include an integrated user interface 212 formed by an operator control, such as a button or key. The external device 222 may provide for an alternative or additional user interface. The sound processor 202 may be connected to an electroacoustic output transducer 110 which may be implemented as a device to be worn in the ear canal. The sound processor 202 may be connected via a connection 208 to a headpiece 206 which comprises a coil for establishing a wireless transcutaneous link 210 with a corresponding coil of the cochlear implant 102. The sound processor 202 may be designed to be worn at the patient's head, for example as a BTE unit; alternatively, it may be integrated within the headpiece. According to another example, the sound processor 202 may be implemented as a body worn unit which is not worn at the head.

The neural response signals detected by the electrodes 106 may be supplied to the sound processor 202 by back-telemetry via the inductive transcutaneous link 210.

What is claimed is:

1. A hearing prosthesis system comprising:
an electrode array configured to be implanted within a patient;
a cochlear implant coupled to the electrode array and configured to be implanted within the patient; and
a processing unit communicatively coupled to the cochlear implant;
wherein the processing unit is configured to:
direct the cochlear implant to apply stimulation to a cochlea of the patient via the electrode array; and
detect, via the electrode array, a neural response of the patient to hearing stimulation;
and wherein the processing unit is further configured to:
generate a user interaction audio signal indicative of an interaction of the patient with the hearing prosthesis system and apply perceivable hearing stimulation to the patient according to the user interaction audio signal, and
record, via the electrode array and the cochlear implant, the neural response to said hearing stimulation according to the user interaction audio signal, thereby utilizing the user interaction audio signal as a test audio signal.

2. The system of claim 1, wherein the user interaction audio signal is a feedback message signal indicative of a user action on a user interface of the hearing prosthesis system or a status message signal indicative of a change in a condition of the hearing prosthesis system.

3. The system of claim 1, wherein the user interaction audio signal is selected such that is perceivable by the patient as a standard sound associated with the respective feedback message or a status message.

4. The system of claim 2, wherein the status message signal is indicative of a wireless connection or disconnection of the processing unit with an external device or of a low battery status.

5. The system of claim 2, wherein the feedback message signal is indicative of a user action resulting in a hearing program change, a volume increase, volume reduction or an implant locking/unlocking.

6. The system of claim 2, wherein the feedback message signal is in response to a user action on an operator control.

7. The system of claim 1, wherein the processing unit is configured to apply the perceivable hearing stimulation corresponding to the user interaction audio signal at a level within a comfort range.

8. The system of claim 1, wherein the user interaction audio signal includes sinusoidal tones and/or frequency sweeps.

9. The system of claim 1, wherein the recording of the neural response to the user interaction audio signal includes neural response imaging (NRI) threshold measurements, electrocochleography (ECochG) threshold measurements, cortical response measurements and/or electrode impedance measurements.

10. The system of claim 1, wherein the processing unit is configured to apply said perceivable hearing stimulation to the patient according to the user interaction audio signal as electrical stimulation via the electrode array.

11. The system of claim 1, wherein the hearing prosthesis system is an EAS system including an electroacoustic output transducer, and wherein the processing unit is configured to apply said perceivable hearing stimulation to the patient according to the user interaction audio signal as acoustic stimulation via the electroacoustic output transducer.

12. The system of claim 11, wherein the recording of the neural response to the user interaction audio signal includes measurement of ECochG signals.

13. The system of claim 1, wherein the processing unit is configured to automatically adjust fitting parameters of the hearing prosthesis system according to the recorded neural response.

14. The system of claim 13, wherein the processing unit is configured to automatically adjust stimulation threshold levels of the hearing prosthesis system according to the recorded neural response, and wherein the user interaction audio signal results in hearing stimulation suitable for determining the respective threshold from the recorded neural response.

15. The system of claim 14, wherein the recording of the neural response to the user interaction audio signal includes cortical response measurements for adjustment of electrical stimulation thresholds.
